(19) United States
(12) Patent Application Publication
(10) Pub. No.: US 2009/0283611 A1
(43) Pub. Date: Nov. 19, 2009

(54) SURFACE TREATMENTS AND COATINGS FOR ATOMIZATION

(75) Inventors: Kripa Kiran Varanasi, Clifton Park, NY (US); Nitin Bhate, Rexford, NY (US); Jeffrey Scott Goldmeer, Latham, NY (US); Geoffrey David Myers, Simpsonville, SC (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 12/120,566

(22) Filed: May 14, 2008

(51) Int. Cl. A61M 11/02

(52) U.S. Cl. 239/366

(57) ABSTRACT

An atomizer, comprising a pre-filming region comprising a surface configured to reduce a mean drop size of a liquid to be atomized, wherein the surface has an effective contact angle, with reference to the liquid, of less than about 30 degrees; and a lip portion disposed at an end of the pre-filming region and configured to create hydrodynamic instabilities in a liquid film, wherein the lip portion comprises an alternating pattern of wetting and non-wetting surfaces, wherein the non-wetting surface comprises a contact angle, with reference to the liquid, of greater than 90 degrees, and the wetting surface comprises a contact angle, with reference to the liquid, of less than 90 degrees.

### BACKGROUND OF THE INVENTION

The present disclosure relates to articles having surfaces engineered to promote selective wetting of the surfaces by liquids. More particularly, this invention relates to enhancing atomization by increasing the wettability of pre-filming region surfaces and inducing hydrodynamic instabilities in selective regions of the atomizer.

Atomization generally refers to the conversion of bulk liquid into a spray or mist (i.e. collection of drops), often by passing the liquid through a nozzle. An atomizer is an apparatus for achieving atomization. Common examples of atomization systems can include: gas turbines, carburetors, airbrushes, misters, spray bottles, and the like. In internal combustion engines for example, fine-grained fuel atomization can be instrumental to efficient combustion.

Current air-blast atomizers spread liquid from a nozzle orifice into a film on one or more pre-filming regions. The atomizers can use pressure, airflow, electrostatic, ultrasonic, and other like methods to create instabilities in the bulk liquid film to form droplets. The bulk liquid film in the pre-filming regions is exposed to high velocity air that enters the regions on both sides of the nozzle orifice. The air streams can create hydrodynamic instabilities in the liquid film and cause it to break up into droplets. The mean drop size generated by an atomizer is significantly influenced by the liquid film uniformity and thickness in its pre-filming region. In some cases the mean drop size can vary with the square root of the film thickness. The thinner the film, therefore, the finer atomization (i.e., the drop size). Current atomizers have no means to ensure that the liquid is spread into the necessary thin film in the pre-filming regions. This can create dry spots on the surface of the pre-filming region that lead to a non-uniform liquid film and, consequently, to larger, coarser droplet sizes.

Therefore, there is a need for improving the uniformity of the bulk liquid film and to introduce further hydrodynamic instabilities for enhancing atomization.

### BRIEF DESCRIPTION OF THE INVENTION

Disclosed herein are atomizers having a surface configured for promoting the atomization of a liquid. In one embodiment the atomizer includes a pre-filming region comprising a surface configured to reduce a mean drop size of an atomized liquid, wherein the surface has an effective contact angle, with reference to the atomized liquid, of less than about 30 degrees.

In another embodiment, an atomizer includes a pre-filming region; and a lip portion disposed at an end of the pre-filming region and configured to create hydrodynamic instabilities in a liquid film, wherein the lip portion comprises an alternating pattern of wetting and non-wetting surfaces, wherein the non-wetting surface comprises a contact angle, with reference to the liquid, of greater than 90 degrees, and the wetting surface comprises a contact angle, with reference to the liquid, of less than 90 degrees.

In still another embodiment, an atomizer is configured to transform a liquid film to a spray, and includes a nozzle for injecting the liquid into a pressurized flow path; a pre-filming region downstream from the nozzle comprising a surface configured to reduce a mean drop size of the liquid, wherein the surface has an effective contact angle, with reference to the liquid, of less than about 30 degrees; and a lip portion disposed downstream of the pre-filming region and configured to create hydrodynamic instabilities in the liquid film, wherein the lip portion comprises an alternating pattern of wetting and non-wetting surfaces, wherein the non-wetting surface comprises a contact angle, with reference to the liquid, of greater than 90 degrees, and the wetting surface comprises a contact angle, with reference to the liquid, of less than 90 degrees.

The above described and other features are exemplified by the following figures and detailed description.

### BRIEF DESCRIPTION OF THE DRAWINGS

Referring now to the figures wherein the like elements are numbered alike:

FIG. 1 is a cross-sectional schematic of a current pre-filming atomizer for use in a combustor system;

FIG. 2 is a cross-sectional schematic view of an exemplary embodiment of the surface of an article showing a coating layer;

FIG. 3 is a cross-sectional schematic view of an exemplary embodiment of the surface of an article showing the texture;

FIG. 4 is a cross-sectional schematic view illustrating the difference between a Wenzel drop state and a Cassie drop state;

FIG. 5 is a plot of effective contact angle as a function of relative spacing for various aspect ratios, where the features are protrusions;

FIG. 6 is the atomizer of FIG. 1 hi-lighting suitable areas for disposing a hybrid texture region;

FIG. 7 illustrates exemplary embodiments of different hybrid patch configurations;

FIG. 8 is a plot of effective contact angle as a function of b/a ratio;

FIG. 9 illustrates exemplary embodiments of hydrophilic Wenzel state surface features;

FIG. 10 shows photographs of oil droplets on silicon post surface features having different b/a ratios to measure roll-off and determine contact angle; and

FIG. 11 is a plot of effective contact angle as a function of relative spacing (b/a ratio) for a hydrophilic/superhydrophillic surface.

### DETAILED DESCRIPTION OF THE INVENTION

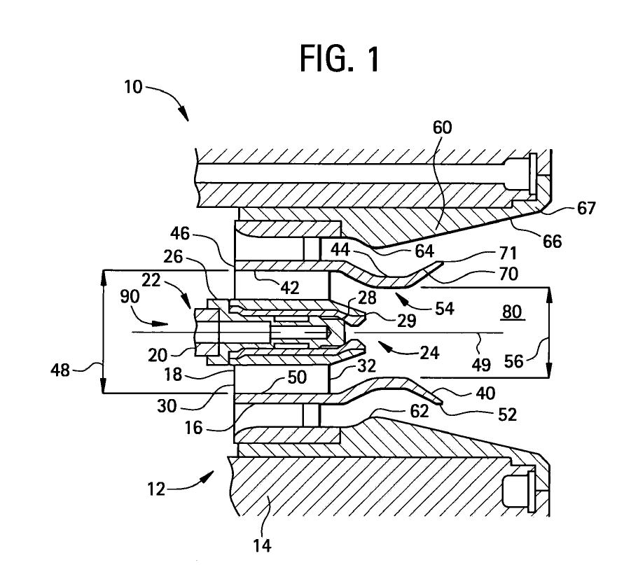

Referring to the drawings in general and to FIG. 1 in particular, it will be understood that the illustrations are for the purpose of describing a particular embodiment of the article disclosed herein and are not intended to be limited thereto. FIG. 1 is a schematic cross-sectional view of an exemplary atomizer of a gas turbine combustor system. Reference herein will be made to the use of surface treatments and coatings in the combustor system. It is to be understood, however, that the surface treatments disclosed herein, can be advantageously used in any atomization system to improve atomizer performance. Examples of systems requiring fluid atomization include, without limitation, agriculture, food preparation, painting, washing, and the like. As described herein, the use of hydrophilic or superhydrophilic surface treatments can result in more uniform, thinner bulk liquid films that can create finer droplet sizes, i.e., reduce the mean drop size of a liquid, when compared to current atomizers without such surface treatments. In addition, the use of hybrid hydrophobic-hydrophillic regions in selective areas, such as the lips of the various pre-filming atomizer regions, can induce hydrodynamic instabilities leading to an efficient breakup of the liquid film and improved atomization.

With regard to combustion applications, such as in turbines, improved atomization can have a significant impact on combustion performance. In general, a measure of air-blast atomizer performance is the air-to-liquid ratio and air-side pressure drop required to produce a spray of a given mean drop size. Typically, the pressure drop is a large fraction of the pressure drop for the entire combustion system, and the air-to-liquid mass flow ratio is equal to or greater than 1.0 for a spray of fine droplet size. In industrial gas turbines or airplane engines, the combustor pressure drop is a penalty (i.e., parasitic loss) to system fuel efficiency. Consequently, the ability to reduce the atomizer pressure drop or air-to-liquid ratio required for a spray of the required quality represents a system-level fuel efficiency benefit. The use of oleophilic and oleophobic or hydrophilic and hydrophobic coatings on the atomizer spray-making surfaces to reduce the mean drop size of the atomized liquid can advantageously result in an improvement in spray quality for a given pressure drop or air-to-liquid ratio relative to an uncoated atomizer. Further, the liquid-wetted portions of the atomizer that produce the spray primarily via pressure-swirl atomization (i.e. at the pre-filming lips) can also benefit from hydrophobic or oleophobic surfaces that permit a lower liquid supply pressure for a given mean droplet size. This reduced pressure can represent a savings in pump work required to supply fuel to the atomization system.

Moreover, the disclosed hydrophobic or oleophobic surfaces can provide the potential benefit of reduced tolerances and precision in manufacturing. The surface treatments can possibly facilitate a more uniform liquid film thickness and distribution on the pre-filming regions, creating a more uniform spray in spite of manufacturing imperfections, such as tooling marks, lack of perfect concentricity, out-of-round condition in metering orifices, and other flaws that would ordinarily cause streaks in the spray and other symptoms of non-uniformity in the fuel film thickness. Even further, the surface treatments disclosed herein could also be used to tailor the spray spatial distribution to better suit the geometry of the combustion system. For example, current nozzles produce an axis-symmetric conical spray, either solid or hollow, depending on the specific type. When injecting this uniform distribution into an annular combustor, there may be more fuel close to the inner and outer walls than desirable from a durability standpoint. The distribution of the disclosed surface treatments on the prefilmer could be used to redistribute the liquid spray, tailoring the mass flux to more evenly distribute the liquid fuel in the downstream combustor volume. In the annular combustor, this could be accomplished using an elliptical cone, rather than a circular cone, with the long axis of the ellipse oriented circumferentially to match the annular volume.

FIG. 1 illustrates an exemplary embodiment of a gas turbine fuel injector 10 of a combustor system. The gas turbine fuel injector 10 can comprise an atomizer 12. The atomizer 12 can include an outer wall 14, a pilot outer swirler 16, a pilot inner swirler 18, and a pilot fuel injector 20. The atomizer 12 has an axis of symmetry 49, and is generally cylindrical-shaped with an annular cross-sectional profile.

Pilot fuel injector 20 is along the axis of symmetry 49 and is positioned within atomizer 12 such that fuel injector is substantially co-axial with atomizer. Fuel injector 20 injects fuel to the pilot and includes an intake side 22, a discharge side 24, and a body 26 extending therebetween. Discharge side 24 includes a convergent discharge nozzle 28, which directs a fuel-flow (not shown) outward from fuel injector 20

Pilot inner swirler 18 is annular and is circumferentially disposed around pilot fuel injector 20. Pilot inner swirler 18 includes an intake side 30 and an outlet side 32. An inner pilot airflow stream (not shown) enters pilot inner swirler intake side 30 and is accelerated prior to exiting through pilot inner swirler outlet side 32.

A baseline air blast pilot splitter 40 is positioned downstream from pilot inner swirler 18. Baseline air blast pilot splitter 40 includes an upstream portion 42 and a downstream portion 44 extending from upstream portion 42. Upstream portion 42 includes a leading edge 46 and has a diameter 48 that is constant from leading edge 46 to air blast pilot splitter downstream portion 44. Upstream portion 42 also includes an inner surface 50 positioned substantially parallel and adjacent pilot inner swirler 18. As used herein the terms “upstream” and “downstream” are intended to describe the location of components within in a combustor system as it relates to the flow of fluid (i.e., fuel) through the system.

Baseline air blast pilot splitter downstream portion 44 extends from upstream portion 42 to a trailing edge 52 of splitter 40. Downstream portion 44 is convergent towards atomizer axis of symmetry 49 such that at a mid-point 54 of downstream portion 44, downstream portion 44 has a diameter 56 that is less than upstream portion diameter 48. Downstream portion 44 diverges outward from downstream portion mid-point 54 such that trailing edge diameter 58 is larger than downstream portion mid-point diameter 56, but less than upstream portion diameter 48.

Pilot outer swirler 16 extends substantially perpendicularly from baseline air blast pilot splitter 40 and attaches to a contoured wall 60. Contoured wall 60 is attached to atomizer outer wall 14. Pilot outer swirler 16 is annular and is circumferentially disposed around baseline air blast pilot splitter 40. Contoured wall 60 includes an apex 62 positioned between a convergent section 64 of contoured wall 60 and a divergent section 66 of contoured wall 60. Splitter downstream portion 44 diverges towards contoured wall divergent section 66. Contoured wall 60 also includes a trailing edge 70 that extends from contoured wall divergent section 66. Trailing edge 70 is substantially perpendicular to atomizer axis of symmetry 49 and is adjacent a combustion zone 80.

In operation, a pilot fuel circuit 90 injects fuel to combustor system 10 through pilot fuel injector 20. Simultaneously, airflow enters pilot swirler intake 30 and is accelerated outward from pilot swirler outlet side 32. The pilot airflow flows substantially parallel to atomizer axis of symmetry 49 and strikes air splitter 40, which directs the pilot airflow in a swirling motion towards fuel exiting pilot fuel injector 20. The pilot airflow does not collapse a spray pattern (not shown) of pilot fuel injector 20, but instead stabilizes and atomizes the fuel.

As the bulk liquid, in this case fuel, exits the pilot fuel injector 20, part of the fuel spreads into a film on surfaces of the atomizer 12. The flat surfaces, for example the inner surface of the pilot splitter or the nozzle lip, can collect a thin film of the fuel and are sometimes known as “pre-filming” surfaces. Such general terminology will be used herein. The flat face of the surface creates a recirculation area of low pressure, which draws the fuel from the discharge nozzle 28 onto the flat surfaces. This “pre-filming” allows a thin layer of the fuel to form. The atomization of the fuel is enhanced by first spreading of the fuel into a thin film layer on the pre-filming surfaces. However, current atomizer pre-filming surfaces are not capable of promoting uniform dispersion of the film across the surface, or thinning of the bulk liquid as it flows along the surfaces downstream (i.e., away from the nozzle). This can lead to non-uniform film thickness causing streaks or gaps in the resulting spray and thicker bulk films that in turn cause coarser droplet sizes and inefficient atomization. By enhancing the wettability of the prefilming surfaces using hydrophilic or superhydrophilic surface treatments, a thinner, more uniform fuel film can form on the surfaces, which can lead to better atomization through finer mean droplet size. Additionally, by treating the lip regions of the pre-filming surfaces with the hybrid hydrophobic-hydrophillic surfaces, hydrodynamic instabilities can be introduced leading to easier film breakage and finer droplet formation.

The gas turbine liquid fuel injector 10 of FIG. 1 is used as an example of identifying pre-filming surfaces for forming bulk liquid films. In an exemplary embodiment, the liquid film can begin by forming on the inner surface 27 of the discharge nozzle 28 and the nozzle lip 29. As more fuel is injected into the combustion zone 80, the fuel can travel downstream and form a film beginning generally at the mid-point 54 of the splitter downstream portion 44 and extend toward to the lip 71 of the trailing edge 70. Likewise, as the fuel continues to be fed through the injector 20, it can spread further out onto the divergent section 66 of the contoured wall 60, advancing toward the wall lip 67. Hydrophilic surface treatments in the flat surface areas, such as on the inner surface 27 of the discharge nozzle 28, the mid-point 54 to the trailing edge 70 of the pilot splitter 40, and the divergent section 66 outward on the contoured wall, can enhance the fuel film uniformity and film thickness by reducing the mean drop size of the fuel. Moreover, a combination of hydrophilic and hydrophobic (i.e. hybrid) surface treatments in strategic areas can create surface hydrodynamic instabilities to significantly improve atomizer performance. Examples of such strategic areas for the hybrid surface treatments can include the nozzle lip 29, the trailing edge lip 71 of the pilot splitter 40, and the contoured wall lip 67. Such treatments can then produce finer and more uniform spray.

The “liquid wettability”, or “wettability,” of a solid surface is determined by observing the nature of the interaction occurring between the surface and a drop of a given liquid disposed on the surface. A surface having a high wettability for the liquid tends to allow the drop to spread over a relatively wide area of the surface (thereby “wetting” the surface). In the extreme case, the liquid spreads into a film over the surface. On the other hand, where the surface has a low wettability for the liquid, the liquid tends to retain a well-formed, ball-shaped drop (the “non-wetting” surface). In the extreme case, the liquid forms spherical drops on the surface that easily roll off of the surface at the slightest disturbance.

The extent to which a liquid is able to wet a solid surface plays a significant role in determining how the liquid and solid will interact with each other. By way of example, so-called “hydrophilic” and “superhydrophilic” materials have relatively high wettability in the presence of water, resulting in a high degree of “sheeting” of the water over the solid surface. Hydrophillic and superhydrophillic surfaces are examples of wetting surfaces. A high degree of wetting results in relatively large areas of liquid-solid contact, and is desirable in applications where a considerable amount of interaction between the two surfaces is beneficial, such as, for example, forming a uniform ultra thin bulk liquid film in an atomizer. One commonly accepted measure of the liquid wettability of a surface is the value of the static contact angle formed between the surface and a tangent to a surface of a droplet of a reference liquid at the point of contact between the surface and the droplet. Low values of the contact angle indicate a high wettability for the reference liquid on surface. The reference liquid may be any liquid of interest. In many applications, the reference liquid is water. In other applications, the reference liquid is a liquid that contains at least one hydrocarbon, such as, for example, oil, petroleum, gasoline, an organic solvent, and the like. Because wettability depends in part upon the surface tension of the reference liquid, a given surface may have a different wettability (and hence form a different contact angle) for different liquids.

The term “hydrophilic” is generally used to describe surfaces that generate, with reference to water, a nominal contact angle of less than about 90 degrees. “Superhydrophilic” is generally used to describe surfaces that generate, with reference to water, a nominal contact angle of less than about 10 degrees. Likewise, the term “hydrophobic” is generally used to describe surfaces that generate, with reference to water, a nominal contact angle of greater than about 90 degrees. “Superhydrophobic” is generally used to describe surfaces that generate, with reference to water, a nominal contact angle of greater than about 150 degrees. Hydrophobic and superhydrophobic surfaces, therefore, are examples of non-wetting surfaces.

In one exemplary embodiment, an atomizer can comprise a pre-filming region comprising a surface configured to reduce a mean drop size of an atomized liquid, wherein the surface has an effective contact angle, with reference to the atomized liquid, of less than about 30 degree. In another exemplary embodiment, an atomizer can have a lip portion disposed downstream of the pre-filming region and configured to create hydrodynamic instabilities in a liquid film, wherein the lip portion comprises an alternating pattern of wetting and non-wetting surfaces, wherein the non-wetting surface comprises a contact angle, with reference to the liquid, of greater than 90 degrees, and the wetting surface comprises a contact angle, with reference to the liquid, of less than 90 degrees. In still another exemplary embodiment, an atomizer can include both the pre-filming region surface described in the first embodiment together with the lip portion hybrid wetting/non-wetting surface described in the second embodiment.

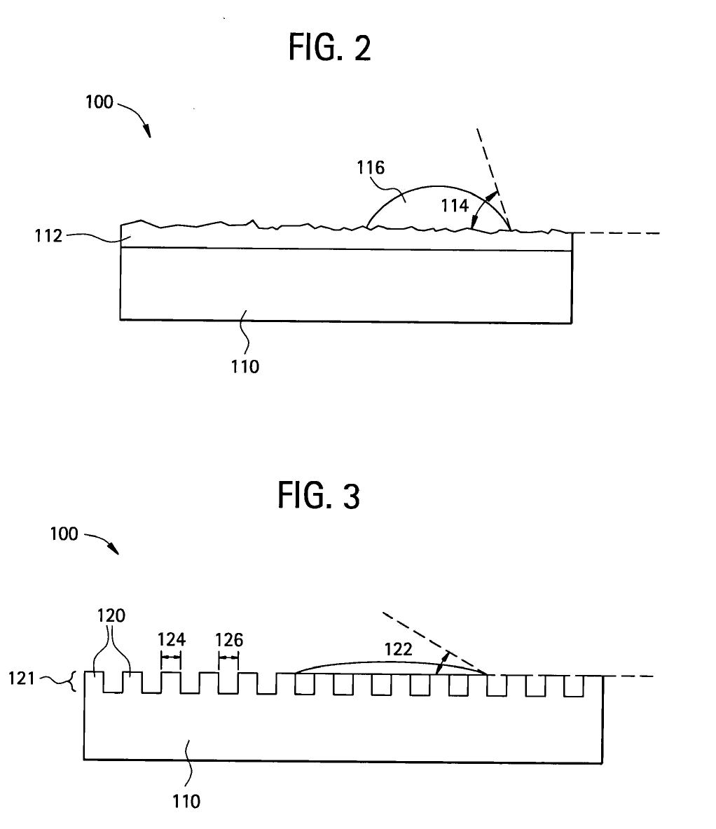

Referring now to FIG. 2, in one exemplary embodiment, the substrate 100 of an atomizer can have a surface 110. The surface 110 could be any of the surfaces described above, where pre-filming would be desirable, such as the inner surfaces of the swirler, the contoured wall, or the discharge nozzle. The surface 110 can comprise a surface energy modification coating layer 112 for modifying the surface energy of the surface. In certain cases, the surface energy modification coating layer 112 can comprise a coating disposed over the surface 110 of the atomizer substrate 100. The surface 110 can comprise at least one of a metal, an alloy, a plastic, a ceramic, or any combination thereof. The surface 110 can take the form of a film, a sheet, or a bulk shape. The coating layer 112 can be an integral part of the surface 110, or the coating layer 112 can comprise a layer that is disposed or deposited onto the surface 110 by any number of techniques that are known in the art.

The surface energy modification coating layer 112 can comprises at least one material selected from a group comprising a hydrophilic coat, such as a ceramic, a composite material, and various combinations thereof. Examples of suitable hydrophilic ceramics include, but are not limited to, inorganic oxides, carbides, nitrides, borides, and combinations thereof. Such ceramic materials include titanium, silicon, aluminum magnesium, zirconium, zinc, yttrium stabilized zirconia, magnesium aluminate spinel, and zinc oxides; aluminum and gallium nitrides; silicon and tungsten carbide cobalt chromium carbide; combinations thereof, and other the like ceramics. The surface material can be selected based on the desired contact angle, the fabrication technique used, and the end-use application of the article. The coating layer materials, and methods for applying them, such as chemical vapor deposition (CVD), physical vapor deposition (PVD), etc., are known in the art, and can be of particular use in harsh environments.

The surface 110 can comprise a coating layer 112 having a nominal wettability sufficient to generate a nominal contact angle of up to about 90 degrees. For better understanding, a “nominal contact angle” 114 means the static contact angle measured where a drop of a reference liquid 116 is disposed on a flat, smooth (<1 nm surface roughness) surface. This nominal contact angle 114 is a measurement of the “nominal wettability” of a material from which the surface is substantially comprised.

In an alternative embodiment, the surface 110 can comprise a plurality of surface features 120, as shown in FIG. 3. The size, shape, and orientation of features 120 have a strong effect on the wettability of surface 110, and in the exemplary embodiments disclosed herein these parameters are selected such that the surface 110 has an effective wettability (that is, wettability of the textured surface) sufficient to generate an effective contact angle less than the nominal contact angle 114 with reference to water. The plurality of surface features 120 can be effective to turn a surface having hydrophilic coating into a surface having superhydrophilic wetabillity.

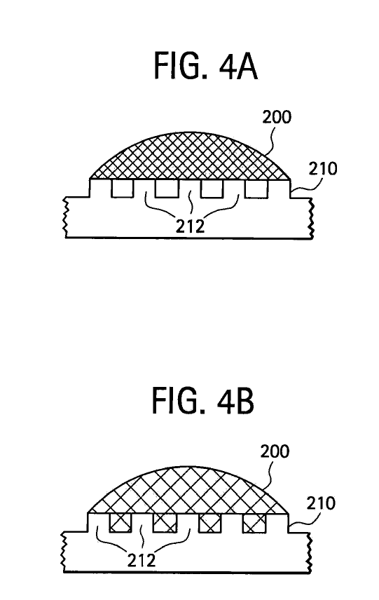

As stated above, the size, shape, and orientation of the features 120 can be selected such that the surface 110 exhibits superhydrophilic wettability. The selection is based upon the physics underlying the interaction of liquids and the solid surfaces. A drop of liquid resides on a textured surface typically in any one of a number of equilibrium states. In the “Cassie” state, depicted in FIG. 4(a), a drop 200 sits on the surface features, in this case posts 212, of the textured surface 210, trapping air pockets between the posts. In the “Wenzel” state, depicted in FIG. 4(b), drop 200 wets the entire surface 210, filling the spaces between the peaks 212 with liquid. Other equilibrium states generally can be envisioned as intermediate states between pure Cassie and pure Wenzel behavior, where drops only partially fill the spaces between surface roughness features. As used herein, the term “non-Wenzel” refers to any state that does not exhibit pure Wenzel-state behavior; as such, the term “non-Wenzel” includes pure Cassie state behavior and any intermediate states that do not exhibit pure Wenzel behavior.

The particular state adopted by the drop on the surface depends on the overall energy of the solid/liquid/vapor system, which in turn is a function of the geometric characteristics—such as the size, shape, and orientation—of the surface roughness features of the solid. For example, where the Cassie state results in a lower energy than the Wenzel state, an impinging drop will generally always exhibit Cassie state behavior. However, even in instances where the Wenzel state provides a lower energy, non-Wenzel state behavior still may be maintained due to the existence of an energy barrier between the two states, requiring the input of energy to achieve the transition from the “metastable” non-Wenzel state to the ultimately lower energy Wenzel state. An understanding of the relationship between surface geometry and energy enables surfaces to be designed to provide desired wettability characteristics, including contact angle and type of wetting state behavior exhibited by liquid on the solid surface.

The effective contact angle theta (θ*) on the textured surface is related to the nominal contact angle (θ) by equation (1) for the Wenzel drop (w) and equation (2) for the Cassie drop (c):

cos(θ_{w}*)=r cos(θ) (1)

cos(θ_{c}*)=f_{SL} cos(θ)−f_{LA} (2)

wherein “r” is the texture parameter and is defined as the contact area for the surface divided by the projected area. For a square array of square posts, r is given by the following expression:

r=1+4(h/a)/(1+b/a)² (3)

where “a” is the width of the posts, “b” is the edge-to-edge spacing between the posts, and “h” is the height of the posts. The expressions for f_{SL} and f_{LA} are given by

f_{SL}=1/(1+b/a)²; and (4)

f_{LA}=1−1/(1+b/a)² (5)

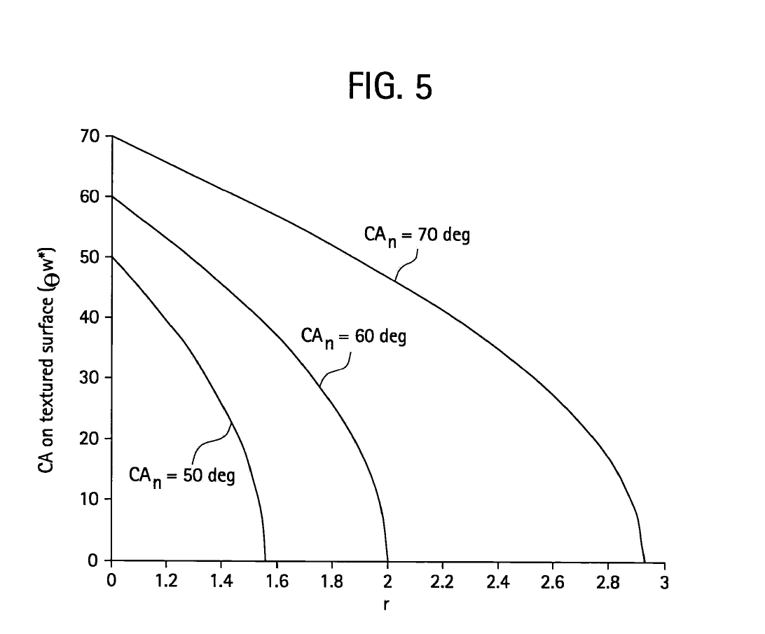

As can be seen by the above expressions, the effective contact angle on textured surfaces is strongly influenced by parameters such as texture feature size, spacing, and aspect ratio. Texturing a surface to produce a Wenzel state droplet is critical to forming a superhydrophilic surface. FIG. 5 is a graph showing the relationship between effective contact angle, θw*, for the Wenzel drop and the texture parameter, r. In this figure, the relationship between the surface area of the surface (as measured by r, the ratio of actual surface area to projected surface area) and the effective contact angle formed with water is plotted for surfaces having nominal contact angles of 50 degrees, 60 degrees, and 70 degrees. It will be apparent that the r parameter is a function of the geometry of the surface; including such parameters as b/a and h/a, and that the nature of the particular function will depend on the configuration of the surface. The graph shows that significant reduction in effective contact angle (as low as 0 degrees) can be achieved in the case of a Wenzel drop when a hydrophilic surface (i.e., a surface with a nominal contact angle of less than 90 degrees) is textured to have surface features. As the texture of the surface features becomes denser (rougher), the value of r increases and the effective contact angle is advantageously lowered. Therefore, when an article has an existing hydrophilic surface (such as most metal surfaces), or the surface has been coated with a hydrophilic surface coating, the surface can be appropriately textured by choosing the texture parameter (r) that results in the desired low or zero effective contact angle by achieving a Wenzel state.

Referring back now to FIG. 3, the size of surface features 120 can be characterized in a number of ways. In some embodiments, as shown in FIG. 3, at least a subset of the plurality of features 120 protrudes from the atomizer substrate 100. Moreover, in some embodiments at least a subset of the plurality of features is a plurality of cavities (not shown) disposed in the atomizer substrate 100. Surface features 120 comprise a height dimension (h) 121, which represents the height of protruding features 120 or, in the case of cavities, the depth to which the cavities extend into atomizer substrate 100. Surface features 120 further comprise a width dimension (a) 124. The precise nature of the width dimension will depend on the shape of the feature, but is defined to be the width of the feature at the point where the feature would naturally contact a drop of liquid placed on the surface of the article. The width, spacing, and height parameters of surface features 120 can have a significant effect on wetting behavior observed on the surface 110.

Numerous varieties of feature shapes are suitable for use as surface features 120. In some embodiments, at least a subset of the surface features 120 has a shape selected from the group consisting of a cube, a rectangular prism, a cone, a cylinder, a pyramid, a trapezoidal prism, and a hemisphere or other spherical portion. These shapes are suitable whether the feature is a protrusion, such as a pedestal, or a cavity, such as a groove or a pore. As an example, in particular embodiments, at least a subset of the features comprises nanowires, which are structures that have a lateral size constrained to tens of nanometers or less and an unconstrained longitudinal size. Methods for making nanowires of various materials are well known in the art, and include, for example, chemical vapor deposition onto a substrate. Nanowires may be grown directly on article 100 or may be grown on a separate substrate, removed from that substrate (for example, by use of ultrasonication), placed in a solvent, and transferred onto article 100 by disposing the solvent onto the article surface and allowing the solvent to dry.

Feature orientation is a further design consideration in the engineering of surface wettability in accordance with embodiments of the present invention. One significant aspect of feature orientation is the spacing of features. Referring to FIG. 3, in some embodiments features 120 are disposed in a spaced-apart relationship characterized by a spacing dimension (b) 126. Spacing dimension 126 is defined as the distance between the edges of two nearest-neighbor features.

In some embodiments, all of the features 120 in the plurality are disposed in a nonrandom distribution. In some cases features 120 have substantially the same respective values for h, a, and/or b (“an ordered array”), though this is not a general requirement. For example, the plurality of features 120 may be a collection of features, such as nanowires, for instance, exhibiting a random distribution of size, shape, and/or orientation. In certain embodiments, moreover, the plurality of features is characterized by a multi-modal distribution (e.g., a bimodal or trimodal distribution) in h, a, b, or any combination thereof. Such distributions may advantageously provide enhanced wettability in environments where a range of drop sizes is encountered. Estimation of the effects of h, a, and b on wettability are thus best performed by taking into account the distributive nature of these parameters. Techniques, such as Monte Carlo simulation, for performing analyses using variables representing probability distributions are well known in the art. Such techniques may be applied in designing features 120 for use in articles as disclosed herein.

Depending upon the application of the atomizer, the article surface 110 can be a material comprised of a metal, such as a metal comprising an element selected from the group consisting of iron, titanium, copper, zirconium, aluminum, and nickel. In certain embodiments the material is essentially completely metallic. In other embodiments, the material comprises a ceramic, such as an oxide typified by titanium oxide, silicon dioxide, and zirconium oxide. Other mildly to very hydrophilic materials, such as, for example, certain polymeric materials, may be used in embodiments of the present invention.

Specific ranges and combinations of the surface feature parameters described above can provide a regime in which the effective wettability of surface 110 may be driven to generate an effective contact angle of less than about 10 degrees with a drop of the reference liquid, in some cases the effective contact angle may be reduced to near zero. Having such a low contact angle on a surface of the pre-filming region of an atomizer can provide for an ultra thin bulk liquid film to form on the surface, which together with current techniques for creating hydrodynamic instabilities in the pre-filming region, will result in finer mean drop size and enhanced atomization.

In an exemplary embodiment, the surface 110 can comprise a plurality of surface features 120 having a median feature size, a, and a median feature spacing, b. The ratio b/a indicates the spacing of the features, and as these features are more closely spaced, the contact area of surface 110 increases (i.e., the texture parameter (r) increases), providing more contact area for the liquid. However, in some situations there is a practical lower limit as to how closely features may be spaced, due in part to limitations in fabrication methods. Moreover, in certain applications, spacing surface features 120 too closely together may cause a situation in which droplets of liquid are suspended between features, without wetting the areas between features 120. Such a condition would reduce the effective wetting area. If (a) changes, but spacing (b) is constant, feature width changes, but feature gap does not change. This, however, could depend on how (b) is defined; is it from feature edge to edge, or feature center to center.

The aspect ratio (h/a) of surface features 120 also plays a role in determining the effective wetting behavior of surface 110. Generally, high aspect ratios, such as at least about 1 and, in some embodiments, at least about 4, are desirable because surface area increases as aspect ratio increases. In some high temperature atomization application, such as, for instance, as found in gas turbines, high aspect ratio (h/a at least about 4) features are desirably sized and spaced apart to give a b/a in the range from about 0.5 to about 6. This combination of parameter values provides a surface that maximizes the coating of an ultra thin uniform film on a atomizer surface.

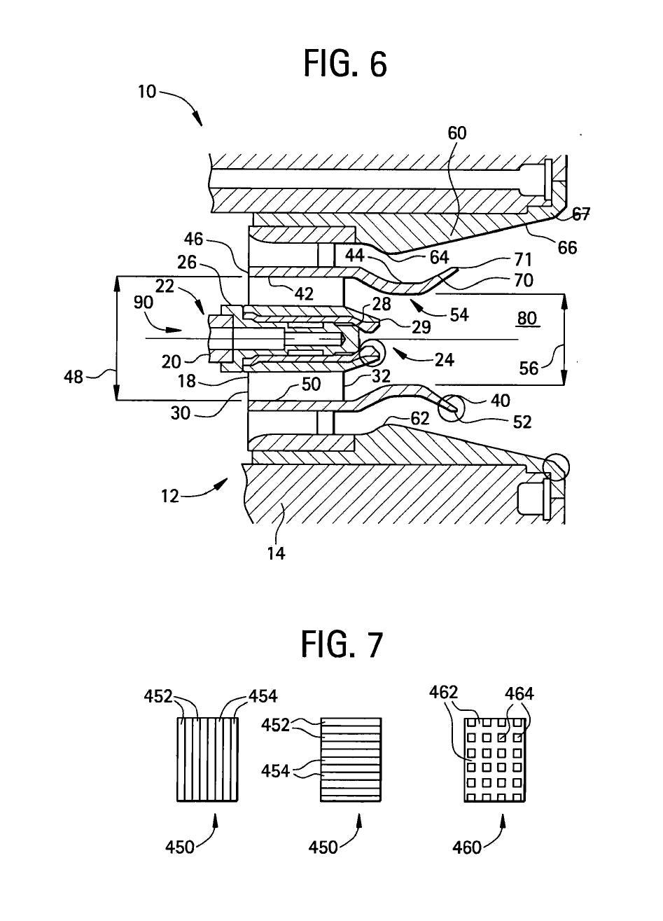

As stated above, beyond having wetting (e.g. hydrophilic or even superhydrophilic) surfaces in the pre-filming regions of an atomizer, further advantages can be achieved when combinations of wetting/non-wetting (e.g. hydrophilic-hydrophobic or “hybrid”) regions are disposed in strategic positions within the atomizer. FIG. 6 illustrates the combustor system and atomizer of FIG. 1. Exemplary locations for the hybrid patches have been circled and pointed out by arrows. In this embodiment, the hybrid patches can be disposed at the nozzle lip 29, the trailing edge lip 71 of the pilot splitter 40, and the contoured wall lip 67. For convenience, these locations will be generally referred to as atomization lips. FIG. 7 illustrates examples of different hybrid patch configurations. It has been discovered that use of the hybrid configurations can induce hydrodynamic instabilities into a bulk liquid film beyond those designed for the atomizer (such as high velocity air). The surface-induced instabilities can work in conjunction with the airflow-induced instabilities to improve the quality of atomization. In an exemplary embodiment, an alternating pattern of hydrophobic (or superhydrophobic) and hydrophilic (or superhydrophilic) texture can be used to create the surface-induced instabilities. For example, as shown in FIG. 7, a hybrid patch 450 can have vertically or horizontally oriented alternating strips of hydrophilic texture 452 and hydrophobic texture 454. In other embodiment, a hybrid patch 460 can form a grid pattern of hydrophobic regions 464 intersected with regions 462 of hydrophilic texture. For example, alternating patches of 'philic-'phobic surface can be done using masking techniques similar to those used in photolithography. Such a method is well known to those in the art, wherein a chemical is used to etch the desired wetting or non-wetting pattern on the surface, while the remainder of the surface is protected by a resistive mask. This same approach can be used not only to create the hybrid patches in two steps, but also to mask the areas of the surface where the coating treatment is not desired. Another method of creating hybrid surfaces is by using micro contact printing.

The strips of hydrophobic texture 454, 464 are areas of low wettability in comparison to the hydrophilic strips 452, 462. Hydrophobic materials have relatively low liquid wettability in order to promote the formation of liquid drops having minimal contact area with the surface 110. Superhydrophobic materials have even lower water wettability, resulting in surfaces that in some cases may seem to repel any water impinging on the surface. The nature of the hydrophobic regions disposed adjacent to hydrophilic regions creates a surface instability in the bulk liquid film at the atomizer lips, because this is where the film start to break up into droplets due to the airflow on the top of the film. By employing the hybrid surfaces in this region, this instability enhances the overall hydrodynamic instability that is desirable in atomization and leads to finer droplet sizes. The edge of the pre-filming lip is a suitable location for creating hydrodynamic instabilities and thinning in the liquid sheet, just prior to the disintegration of the sheet into ligaments and drops.

The regions of hydrophobic surface can have a texture comprising a plurality of features just like the hydrophilic surfaces described above. The surface features, however, have shapes and parameters better suited to providing a surface with lower effective wetabillity than the nominal wettability inherent to the material from which the surface is made. The surfaces thus designed and fabricated have a selected wettability for water and oil to create surface instabilities in atomizer lip areas of the combustor system 10. In one embodiment, the nominal contact angle, with reference to water, is greater than about 100 degrees, specifically greater than about 120 degrees, and more specifically greater than about 150 degrees.

In exemplary embodiments, the regions of hydrophobic surface 454,464 comprise surface textures having high contact angle (low wettability) for water and oil, and also easy drop roll-off. Through proper selection of b/a, and h/a, coupled with proper selection of materials based on the application environment, a surface can be designed such that drops of liquid impinging on the surface will exhibit hydrophobic and oil resistant properties combined with easy roll-off behavior. Accordingly, the surface features comprise a height dimension (h), a width dimension (a), and a spacing dimension (b) such that the ratio b/a is less than about 4, and ratio h/a is less than about 10. In an exemplary embodiment, parameter a is less than about 25 micrometers, specifically less than about 10 micrometers, and more specifically less than about 2 micrometers. In some embodiments, b/a can be in a range from about 0.3 to about 10, specifically about 0.5 to about 2; and h/a can be in a range from about 0.5 to about 5, specifically about 0.5 to about 1.

The surface features for both the hydrophilic surface 110 and the hybrid configuration patches 450, 460 can be fabricated and provided to the atomizer substrate 100 by a number of methods. In some embodiments, the surface features can be fabricated directly on surface 110. In other embodiments, surface features can be fabricated separately and then disposed onto the substrate 100. Disposition of the surface features onto the substrate 100 can be done by individually attaching the features, or the features can be disposed on a sheet, foil or other suitable medium that is then attached to the substrate 100. Attachment in either case may be accomplished through any appropriate method, such as, but not limited to, welding, brazing, mechanically attaching, or adhesively attaching via epoxy or other adhesive, thermal spraying, and the like.

The disposition of surface features may be accomplished by disposing material onto the surface of the article, by removing material from the surface, or a combination of both depositing and removing. Many methods are known in the art for adding or removing material from a surface. For example, simple roughening of the surface by mechanical operations such as grinding, grit blasting, shot peening, and the like, may be suitable if appropriate media/tooling and surface materials are selected. Such operations will generally result in a distribution of randomly oriented features on the surface, while the size-scale of the features will depend significantly on the size of the media and/or tooling used for the material removal operation. General roughening of surfaces to promote enhanced wetting can be used to create surface features. However, certain embodiments of the present invention require control over specific parameters such as relative spacing and aspect ratio of the surface features to provide improved or lowered wetting performance. Many of the parameter ranges and combinations thereof are very difficult or impossible to achieve via the use of traditionally described roughening processes such as grit blasting, for example.

Lithographic methods are commonly used to create surface features on etchable surfaces, including metal surfaces. Ordered arrays of features can be provided by these methods; the lower limit of feature size available through these techniques is limited by the resolution of the particular lithographic process being applied. Lithography and other etching methods are generally not well-suited to the formation of high aspect ratio features on some metal surfaces, however, due to the tendency to “undercut,” i.e., to etch laterally as well as vertically.

Electroplating methods are also commonly used to add features to surfaces. An electrically conductive surface may be masked in a patterned array to expose areas upon which features are to be disposed, and the features may be built up on these exposed regions by plating. This method allows the creation of features having higher aspect ratios than those commonly achieved by etching techniques. In particular embodiments, the masking is accomplished by the use of an anodized aluminum oxide (AAO) template having a well-controlled pore size. Material is electroplated onto the substrate through the pores, and the AAO template is then selectively removed; this process is commonly applied in the art to make high aspect ratio features such as nanorods. Nanorods of metal and metal oxides may be deposited using commonly known processing, and these materials may be further processed (by carburization, for example) to form various ceramic materials such as carbides. As will be described in more detail below, coatings or other surface modification techniques may be applied to the features to provide even better wettability properties.

Micromachining techniques, such as laser micromachining (commonly used for silicon and stainless steels, for example) and etching techniques (for example, those commonly used for silicon) are suitable methods as well. Such techniques may be used to form cavities (as in laser drilling) as well as protruding features. Where the plurality of surface features includes cavities, in some embodiments the article can comprise a porous material, such as, for example, an anodized metal oxide. Anodized aluminum oxide is a particular example of a porous material that may be suitable for use in some embodiments. Anodized aluminum oxide typically comprises columnar pores, and pore parameters such as diameter and aspect ratio may be closely controlled by the anodization process, using process controls that are well known to the art to convert a layer of metal into a layer of porous metal oxide.

Brazing techniques can be used to attach surface features to the article. In this method, a coating mixture can be deposited on the surface of the article substrate, wherein the coating mixture can comprise a braze material and a texture-providing material. The braze material can then be heated to bond the texture-providing material to the surface of the article. In another method, the surface features can added via a thermal-spray or cold-spray process. For example, a mixture of particles (nano-sized to micro-sized) and a binder can be deposited onto the surface of the article substrate to for a hydrophilic or hydrophobic surface. The mixture can be deposited without melting of the particles to ensure the proper texture of the surface.

In short, any of a number of deposition processes or material removal processes commonly known in the art may be used to provide features to a surface. As described above, the surface features may be applied directly onto substrate 100, or applied to a substrate that is then attached to the substrate 100.

The nature of the application will determine the extent to which features are to be disposed on an article. Non-uniform film layers that are thicker than desired result in combustor inefficiency and increased fuel consumption and cost. Atomizers having surfaces configured for promoting atomization of a liquid as disclosed herein can improve the uniformity and droplet size of spray and increase the efficiency of combustor systems. The aforementioned embodiments present clear advantages over existing atomization systems and turbine components comprising such surfaces. In addition, these atomization surfaces may improve the performance of fuel vaporizers. These devices generate fuel vapor, which can then be mixed with an inert gas or steam so that fuel can be burned in premixers designed for gaseous fuel injection. Fuel vaporizers may become an important means of consuming liquid fuels in existing dry, low emissions combustor systems, without developing a secondary spray combustion capability. The improved atomization provided by the surface treatments and coatings as disclosed herein could reduce the required heat input into a fuel vaporizer and improve vaporizer efficiency.

The following example serves to illustrate the features and advantages offered by the embodiments of the present disclosure and are not intended to be limiting thereto.

### EXAMPLES

### Example 1

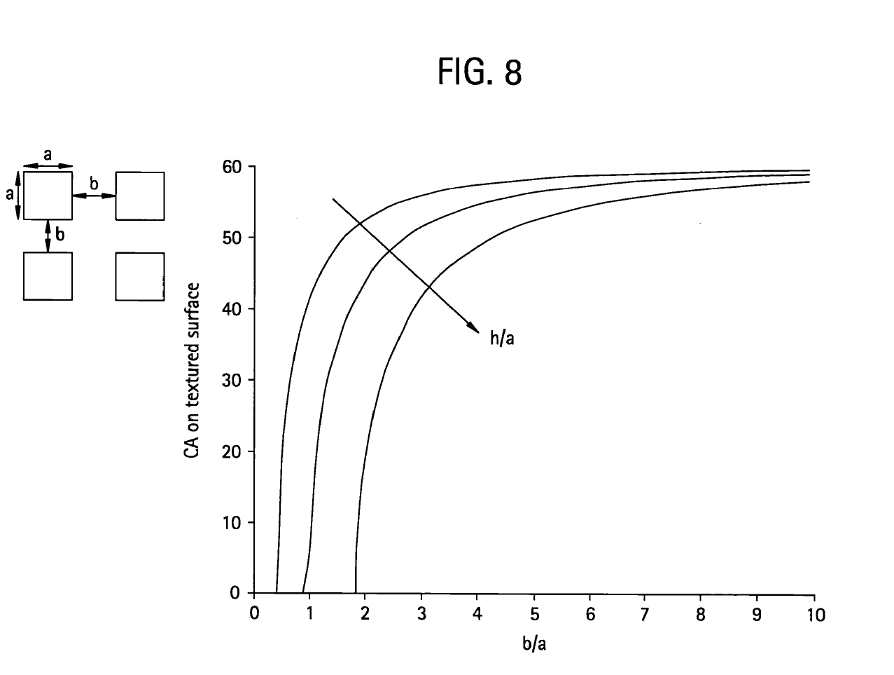

Silicon substrates were provided via lithography with right rectangular prism post features about 3 micrometers in width (a) and having a variety of post spacings (b/a ratios) and aspect ratios (h/a). The substrates were then placed in a chamber with a vial of liquid fluorsilane (FS), and the chamber was evacuated to allow the liquid to evaporate and condense from the gas phase onto the silicon substrate, thereby creating a hydrophilic film on the surface. The effective Wenzel state contact angle was recorded as a function of b/a ratio. FIG. 8 graphically illustrates the trend of varying the b/a ratio to reduce the effective contact angle. Three surfaces having nominal contact angles (CAₙ) of 50, 60, and 70 degrees respectively were textured with the rectangular post surface features. As shown in FIG. 8, the effective contact angle is lowered (as low as about zero degrees) as the relative spacing between each post is reduced. Moreover, increasing the aspect ration can result in lowering of the contact angle for the same spacing because it is energentically favorable to wet more area. Measurements on silicon wafers with square pedestals show that for a certain range of b/a the contact angle is as low as zero degrees. These results are shown below in Table 1 where the surface features parameters (height, width, and spacing) generate about a zero degree effective contact angle.

TABLE 1

| a | b | Aspect Ratio | Effective Contact Angle |
|---|---|---|---|
| (um) | (um) | (h/a) | (degrees) |
| 3 | 1 | 3.3 | 0 |
| 3 | 1.5 | 3.3 | 0 |
| 3 | 2 | 3.3 | 0 |
| 3 | 3 | 3.3 | 0 |
| 3 | 4.5 | 3.3 | 0 |
| 3 | 6 | 3.3 | 0 |
| 15 | 5 | 1.6 | 0 |
| 15 | 7.5 | 1.6 | 0 |
| 15 | 15 | 1.6 | 0 |
| 15 | 11.3 | 1.6 | 0 |

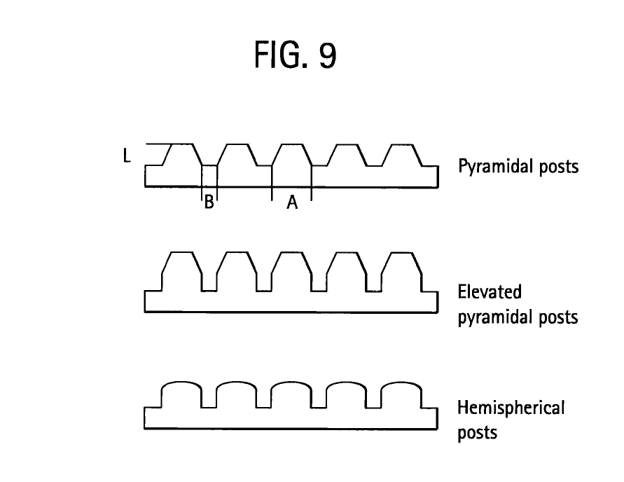

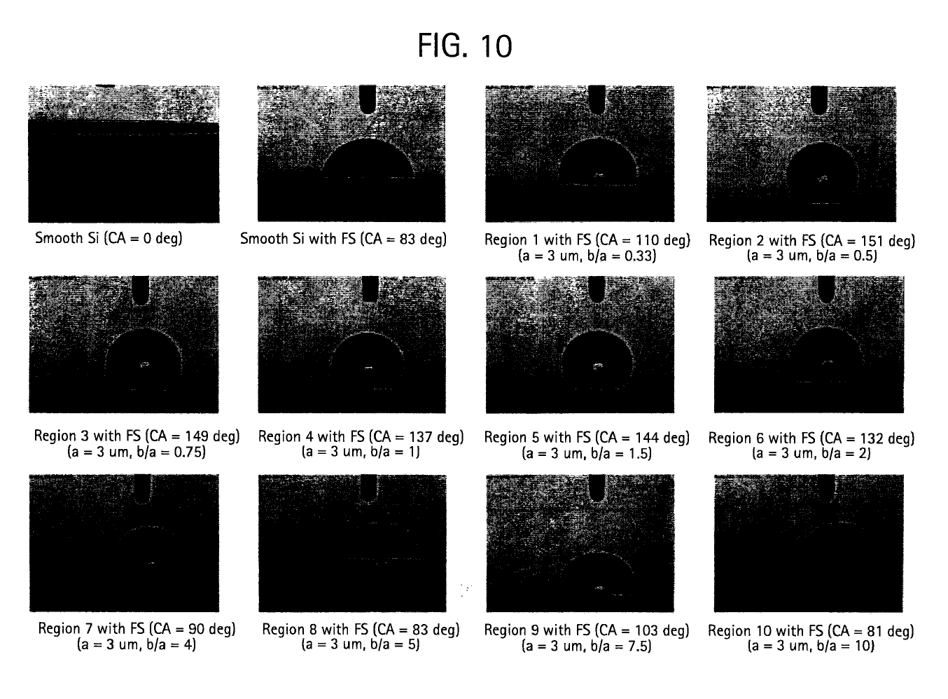

FIG. 9 further shows examples of the post surface features configured to form Wenzel state drops and to generate the low effective contact angles shown in the table above. The top example illustrates post features having a pyramidal top surface. The middle example illustrates post features having an elevated pyramidal top surface, wherein the features have a large height dimension than the top example and the pyramid shape does not cover the entire feature. Finally, the bottom example illustrates post features with a hemispherical top surface.

FIG. 10 shows the photographs of oil droplets on silicon posts with different b/a ratios. FIG. 10 lists the nominal contact angle of oil on different surface features parameters. The oil used was engine lube oil terasitic GT 32® commercially available from Exxon Mobil. The surfaces are generally oleophobic in nature. The ease of roll-off was measured by determining the angle of tilt from the horizontal needed before a drop will roll off of a surface. A drop that requires a near vertical tilt is highly pinned to the surface, whereas a drop exhibiting easy roll-off will require very little tilt angle to roll off the surface. Regions 5 and 6 were the only regions where oil droplets rolled off the posts. The oil droplets were 2 and 4 microliters in volume. For comparison, the same features were tested with water droplets of similar volume. With water as a reference liquid, the droplets rolled off regions 5 through 10. Based on the roll-off date on the smooth silicon wafers coated with FS, the pinning parameter was calculated to be 0.029 Newtons per meter (N/m). The parameter was tested using a goniometer and calculated based on the equation: ρVg sin θ=μl, wherein ρ is density of liquid, V is the volume of drop, g is gravity, θ is the contact angle, μ is the pinning parameter, and 1 is the contact line length. For water, the pinning parameter is on the order of 0.013 N/m. From this data, it can be seen that a different surface feature design will be needed for oleophobic surface applications as compared to hydrophobic surfaces.

### Example 2

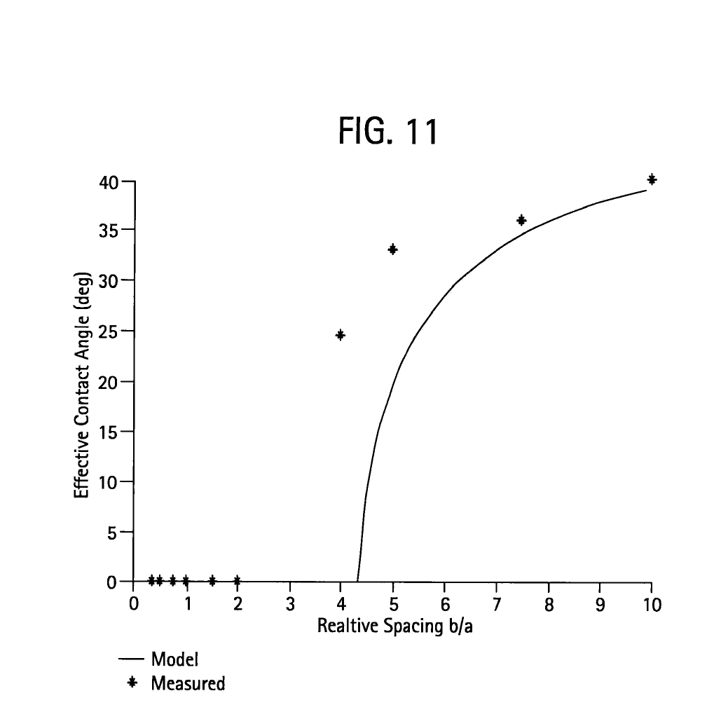

FIG. 11 is a plot of effective contact angles (degrees) versus relative spacing of surface features (spacing dimension (b) divided by width dimension (a)). The graph shows a variety of hydrophilic/superhydrophillic surfaces that could be employed in the pre-filming and/or lip regions of an atomizer. The surface features were posts protruding from the surface and had a width dimension (a) of about 3 micrometers. As seen in the figure, the effective contact angle of the surface increased from about 25 degrees to about 40 degrees when the relative spacing of the post features was increased from about 4 to about 10. When the relative spacing of the features were closer (e.g. a b/a to of less than about 4), the effective contact angle of the surface was about 0 degrees or completely wetting.

Ranges disclosed herein are inclusive and combinable (e.g., ranges of “up to about 25 wt %, or, more specifically, about 5 wt % to about 20 wt %”, is inclusive of the endpoints and all intermediate values of the ranges of “about 5 wt % to about 25 wt %,” etc.). “Combination” is inclusive of blends, mixtures, alloys, reaction products, and the like. Furthermore, the terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by context, (e.g., includes the degree of error associated with measurement of the particular quantity). The suffix “(s)” as used herein is intended to include both the singular and the plural of the term that it modifies, thereby including one or more of that term (e.g., the colorant(s) includes one or more colorants). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

While the invention has been described with reference to a preferred embodiment, it will be understood that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An atomizer, comprising:
a pre-filming region comprising a surface configured to reduce a mean drop size of an atomized liquid, wherein the surface has an effective contact angle, with reference to the atomized liquid, of less than about 30 degrees.

2. The atomizer of claim 1, wherein the surface comprises a surface energy modification coating layer.

3. The atomizer of claim 2, wherein the surface energy modification coating layer comprises a ceramic material, a hydrophilic polymeric material, or a combination comprising at least one of the foregoing materials; wherein the ceramic material comprises titanium oxide, silicon oxide, aluminum oxide, magnesium oxide, zirconium oxide, zinc oxide, yttrium stabilized zirconia, magnesium aluminate spinel, aluminum nitride, gallium nitride, silicon carbide, tungsten carbide cobalt chromium, or a combination comprising at least one of the foregoing.

4. The atomizer of claim 1, wherein the surface comprises a textured pattern, wherein the textured pattern comprises a plurality of surface features having a height dimension (h), a width dimension (a), and a spacing dimension (b), wherein a ratio of b to a (b/a) is less than or equal to 8, and wherein the plurality of surface features have an effective contact angle, with reference to the atomized liquid, of less than about 30 degrees.

5. The atomizer of claim 4, wherein the plurality of surface features comprises a plurality of posts protruding about the surface, wherein each of the posts comprise a width dimension (a) less than about 100 micrometers and an aspect ratio (h/a) greater than about 0.25.

6. The atomizer of claim 4, wherein the plurality of surface features comprises a plurality of pores disposed on the surface, wherein each of the pores comprise a width dimension (a) less than about 100 micrometers and an aspect ratio (h/a) greater than about 0.25.

7. An atomizer, comprising:
a pre-filming region; and
a lip portion disposed at an end of the pre-filming region and configured to create hydrodynamic instabilities in a liquid film, wherein the lip portion comprises an alternating pattern of wetting and non-wetting surfaces, wherein the non-wetting surface comprises an effective contact angle, with reference to the liquid, of greater than 90 degrees, and the wetting surface comprises a contact angle, with reference to the liquid, of less than 90 degrees.

8. The atomizer of claim 7, wherein a selected one or both of the wetting surfaces and the non-wetting surfaces comprise a surface energy modification coating layer.

9. The atomizer of claim 8, wherein the wetting surfaces comprise the surface energy modification coating layer, wherein the layer comprises a ceramic material, a hydrophilic polymer material, or a combination comprising at least one of the foregoing materials; wherein the ceramic material comprises titanium oxide, silicon oxide, aluminum oxide, magnesium oxide, zirconium oxide, zinc oxide, yttrium stabilized zirconia, magnesium aluminate spinel, aluminum nitride, gallium nitride, silicon carbide, tungsten carbide cobalt chromium, or a combination comprising at least one of the foregoing.

10. The atomizer of claim 8, wherein the non-wetting surfaces comprise the surface energy modification layer, wherein the layer comprises at least one material selected from the group consisting of a ceramic, a polymeric, a fluorinated material, an intermetallic compound, and a composite material, wherein the ceramic comprises diamond-like carbon, fluorinated diamond-like carbon, tantalum oxide, titanium carbide, titanium nitride, chromium nitride, boron nitride, chromium carbide, molybdenum carbide, titanium carbonitride, electroless nickel, zirconium nitride, silicon dioxide, titanium dioxide, or a combination comprising at least one of the foregoing; wherein the intermetallic compound comprises nickel aluminide, titanium aluminide, or a combination comprising at least one of the foregoing; and wherein the polymeric material comprises polytetrafluoroethylene, fluoroacrylate, fluoroeurathane, fluorosilicone, fluorosilane, modified carbonate, silicone, or a combination comprising at least one of the foregoing.

11. The atomizer of claim 7, wherein the wetting surfaces comprise a textured pattern, wherein the textured pattern comprises a plurality of surface features having a height dimension (h), a width dimension (a), and a spacing dimension (b), wherein a ratio of b to a (b/a) is less than or equal to 8, and wherein the plurality of surface features have an effective contact angle, with reference to the liquid, of less than about 80 degrees.

12. The atomizer of claim 11, wherein the plurality of surface features are a selected one or both of a plurality of posts and a plurality of pores, wherein each of the post and pores have a width dimension (a) of less than about 100 micrometers and an aspect ration (h/a) of greater than about 0.25.

13. The atomizer of claim 11, wherein the effective contact angle is less than about 30 degrees.

14. The atomizer of claim 11, wherein the effective contact angle is less than about 10 degrees.

15. The atomizer of claim 7, wherein the non-wetting surfaces comprise a textured pattern, wherein the textured pattern comprises a plurality of surface features having a height dimension (h), a width dimension (a), and a spacing dimension (b), wherein a ratio of b to a (b/a) is less than or equal to 8, and wherein the plurality of surface features have an effective contact angle, with reference to the atomized liquid, of greater than about 100 degrees.

16. The atomizer of claim 15, wherein the plurality of surface features are a selected one or both of a plurality of posts and a plurality of pores, wherein each of the post and pores have a width dimension (a) of less than about 100 micrometers and an aspect ration (h/a) of greater than about 0.25.

17. The atomizer of claim 15, wherein the effective contact angle is greater than about 120 degrees.

18. The atomizer of claim 15, wherein the effective contact angle is greater than about 150 degrees.

19. An atomizer configured to transform a liquid film to a spray, the atomizer comprising:
a nozzle for injecting the liquid into a pressurized flow path;
a pre-filming region downstream from the nozzle comprising a surface configured to reduce a mean drop size of the liquid, wherein the surface has an effective contact angle, with reference to the liquid, of less than about 30 degrees; and
a lip portion disposed downstream of the pre-filming region and configured to create hydrodynamic instabilities in the liquid film, wherein the lip portion comprises an alternating pattern of wetting and non-wetting surfaces, wherein the non-wetting surfaces comprise an effective contact angle, with reference to the liquid, of greater than 90 degrees, and the wetting surfaces comprise an effective contact angle, with reference to the liquid, of less than 90 degrees.

20. The atomizer of claim 19, wherein a selected one or both of the wetting and non-wetting surfaces comprise a textured pattern, wherein the textured pattern comprises a plurality of surface features having a height dimension (h), a width dimension (a), and a spacing dimension (b), wherein a ratio of b to a (b/a) is less than or equal to 8; and wherein the plurality of features comprise a selected one or both of a plurality of posts and a plurality of pores, wherein the plurality of posts protrude above the surface and the plurality of pores are disposed on the surface.
